(19) United States
(12) Patent Application Publication
(10) Pub. No.: US 2016/0128992 A1
(43) Pub. Date: May 12, 2016

(54) Methods for limiting acute kidney injury

(71) Applicant: VANDERBILT UNIVERSITY, Nashville, TN (US)

(72) Inventors: Billy G. HUDSON, Nashville, TN (US); Paul VOZIYAN, Nashville, TN (US); Mark deCAESTECKER, Nashville, TN (US); Nataliya SKRYPNYK, Nashville, TN (US)

(21) Appl. No.: 14/936,905

(22) Filed: Nov. 10, 2015

Related U.S. Application Data
(60) Provisional application No. 62/078,299, filed on Nov. 11, 2014.
(60) Provisional application No. 62/130,435, filed on Mar. 9, 2015.
(60) Provisional application No. 62/169,996, filed on Jun. 2, 2015.

(51) Int. Cl. A61K 31/4415; C12Q 1/68

(52) U.S. Cl. CPC A61K 31/4415; C12Q 1/6883; C12Q 2600/158; C12Q 2600/106

(57) ABSTRACT

Method of limiting development of acute kidney injury (AKI) and treating AKI using pyridoxamine are described, together with methods for monitoring efficacy of pyridoxamine therapy.

### CROSS REFERENCE

This application claims priority to U.S. Provisional Patent Application Ser. No. 62/078,299 filed Nov. 11, 2014; 62/130,435 filed Mar. 9, 2015; and 62/169,996 filed Jun. 2, 2015, each incorporated by reference herein in their entirety.

### BACKGROUND

Acute kidney injury (AKI)—also called acute renal/kidney failure—develops rapidly over a period of a few hours or days. AKI can lead to chronic kidney disease (CKD), or even kidney failure needing dialysis (end-stage kidney disease). It may also lead to heart disease or death.

### SUMMARY OF THE INVENTION

In a first aspect, the invention provides methods of limiting development of acute kidney injury (AKI), comprising administering to a subject to be subjected to a precipitating event an amount effective of pyridoxamine, or a pharmaceutically acceptable salt thereof, to limit development of the AKI, wherein the administering comprises administering pyridoxamine, or a pharmaceutically acceptable salt thereof, to the subject prior to, at the time of, or within 24 hours of the precipitating event. In another aspect, the invention provides methods of limiting development of acute kidney injury (AKI), comprising administering to a subject at risk of AKI an amount effective of pyridoxamine, or a pharmaceutically acceptable salt thereof, to limit development of the AKI. In a further aspect, the invention provides methods of treating development of acute kidney injury (AKI), comprising administering to a subject with AKI an amount effective of pyridoxamine, or a pharmaceutically acceptable salt thereof, to treat AKI. In a still further aspect, the invention provides methods for monitoring efficacy of pyridoxamine therapy, comprising

(a) determining one or more of the following in a biological sample obtained from a subject receiving pyridoxamine therapy (a) expression level of Col3α1, (b) expression level of αSMA, (c) expression level of Kim1, (d) expression level of NGAL, (e) expression level of Col1α1, and/or (e) isofuran-to-isoprostane ratio (IsoF/IsoP); and

(b) comparing the levels of markers determined in step (a) to a control;

wherein those subjects with a decreased level of one or more of the markers compared to control are responding to pyridoxamine therapy.

### DESCRIPTION OF THE FIGURES

FIG. 1. Dose dependent effects of pre-treatment with pyridoxamine (PYR) at 500 and 1000 mg/kg/day on renal fibrosis 28 days after I/R-AKI. (A) Experimental model. Mice underwent unilateral renal pedicle clamping (U-IR) followed by contralateral nephrectomy 8 days after the initial surgery. All mice were pre-treated for 3 days with either vehicle control, or PYR 500 and 1000 mg/kg/day in drinking water supplemented with 200 mg PYR twice a day (or vehicle) by oral gavage for 3 days after each surgical procedure. Treatment was continued for 28 days at which point mice were sacrificed and kidney harvested for analysis. (B-D) Expression of renal fibrosis markers Col1α1, α-SMA and Col3α1 mRNA relative to Gapdh mRNA control. (E) Quantification of Sirius red stained (% total area). (F) Representative images for Sirius red stained tissues (outer medulla; scale bars, 50 μm). Results expressed as mean+/−SEM, n=9-10 mice per group. Results only indicated if ANOVA p<0.05: *p<0.05, **p<0.01, ***p<0.001, #p<0.0001. Comparison with uninjured controls (no brackets), or vehicle treated mice (brackets).

FIG. 2. Dose dependent effects of pre-treatment with PYR at 500 and 1000 mg/kg/day on markers of renal injury 28 days after I/R-AKI. Effect of PYR on Kim1 (A) and NGAL (B) mRNA on day 28 after injury. Results expressed as mean+/−SEM, n=9-10 mice per group. Results only indicated if ANOVA p<0.05: *p<0.05, **p<0.01, ***p<0.001, #p<0.0001. Comparison with uninjured controls (no brackets), or vehicle treated mice (brackets).

FIG. 3. Beneficial effects of treatment with PYR at 1000 mg/kg/day started 24 hours after injury on renal fibrosis 28 days after I/R-AKI. (A) Experimental model. Mice underwent U-IR followed by contralateral nephrectomy 8 days after the initial surgery. Mice were treated with PYR 1000 mg/kg/day starting 24 hours after the initial injury supplemented with 200 mg PYR twice a day (or vehicle) by oral gavage for 3 days after each surgical procedure. Treatment was continued for 28 days at which point mice were sacrificed and kidney harvested for analysis. (B-D) Expression of renal fibrosis markers Col1α1, α-SMA and Col3α1 mRNA relative to Gapdh mRNA control. (E) Quantification of Sirius red stained (% total area). (F) Representative images for Sirius red stained tissues (outer medulla; scale bars, 50 μm). Results expressed as mean+/−SEM, n=8-10/group. Results indicated if ANOVA p<0.05: *p<0.05, **p<0.01, ***p<0.001, #p<0.0001. Comparison with uninjured (no brackets), or vehicle or delayed PYR treatment (brackets).

FIG. 4. No effects of treatment with PYR at 1000 mg/kg/day started 24 hours after injury on markers of renal injury 28 days after I/R-AKI. (A, B) Expression of renal injury markers Kim1 and NGAL mRNA relative to Gapdh mRNA control on day 28 after injury. Results expressed as mean+/−SEM, n=8-10/group. Results indicated if ANOVA p<0.05: *p<0.05, **p<0.01, ***p<0.001, #p<0.0001. Comparison with uninjured (no brackets), or vehicle or delayed PYR treatment (brackets).

FIG. 5. Dose dependent effect of pre-treatment with PYR at 500 and 1000 mg/kg/day on renal injury 3 days after I/R-AKI. (A) Experimental model. Mice underwent U-IR and were pre-treated for 3 days with either vehicle control, PYR 500 mg/kg/day or PYR 1000 mg/kg/day in drinking water supplemented with 200 mg PYR twice a day (or vehicle) by oral gavage for 3 days after the surgical procedure. Mice were sacrificed and kidney harvested for analysis 3 days after the initial injury. (B, C) Renal Kim1 and NGAL mRNA expression 3 days after injury expressed as the ratio to Gapdh mRNA control. (D) Tubular injury score 3 days after injury in the OM (0-4, arbitrary units). (E) Representative images for PAS stained tissues (outer medulla; scale bars, 50 μm) F) Expression renal isofuran/isoprostane ratios after PYR treatment with 500 and 1000 mg/kg/day 3 days after U-IR. Results expressed as mean+/−SEM, n=9-10 mice per group. Results only indicated if ANOVA p<0.05: *p<0.05, **p<0.01, ***p<0.001, #p<0.0001. Comparison with uninjured (no brackets), or vehicle or PYR 500 mg/kg/day treated mice (brackets).

FIG. 6. Plasma PYR levels after I/R-AKI. (A) Mice underwent U-IR and were pre-treated for 3 days with vehicle control, PYR 500 mg/kg/day or PYR 1000 mg/kg/day in drinking water supplemented with 200 mg PYR twice a day (or vehicle) by oral gavage for 3 days after the surgical procedure. Evaluation of PYR plasma levels on Day 3 after injury. (B) Mice underwent U-IR followed by contralateral nephrectomy 8 days after the initial surgery. All mice were pre-treated with vehicle control, PYR 500 mg/kg/day or 1000 mg/kg/day in drinking water supplemented with 200 mg PYR twice a day (or vehicle) by oral gavage for 3 days after each surgical procedure. Treatment was continued for 28 days at which point mice underwent venesection for analysis of plasma PYR levels. Evaluation of PYR plasma levels on Day 28 after injury. Results expressed as mean+/−SEM, n=9-10 mice per group. Results only indicated if ANOVA p<0.05: *p<0.05, **p<0.01, ***p<0.001, #p<0.0001. Comparison with uninjured controls (no brackets), or vehicle or PYR 500 mg/kg/day treated mice (brackets).

### DETAILED DESCRIPTION OF THE INVENTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise. “And” as used herein is interchangeably used with “or” unless expressly stated otherwise.

All embodiments of any aspect of the invention can be used in combination, unless the context clearly dictates otherwise.

In a first aspect, the invention provides methods of limiting development of acute kidney injury (AKI), comprising administering to a subject to be subjected to a precipitating event an amount effective of pyridoxamine, or a pharmaceutically acceptable salt thereof, to limit development of the AKI, wherein the administering comprises administering pyridoxamine, or a pharmaceutically acceptable salt thereof, to the subject prior to, at the time of, or within 12 hours of the precipitating event.

An “acute kidney injury” (AKI) refers to an abrupt loss of kidney function that develops shortly after a precipitating event; for example, a loss of kidney function that occurs within 7 days of a precipitating event. For example, AKI may be diagnosed once a subject experiences one or more of:
a twofold increase in serum creatinine,
a glomerular filtration rate (GFR) decrease by 50 percent,
urine output <0.5 mL/kg per hour for 12 hours.

A “precipitating event” is any occurrence or risk factor that leads to AKI. In various non-limiting embodiments, the precipitating event may be a disease or a medical procedure. In one embodiment, the precipitating event may be a medical procedure that can result in reduced effective blood flow to the kidney, including but not limited to cardiovascular surgery. In another embodiment, the precipitating event may be injection of a contrast dye for medical imaging or other purposes. In a further embodiment, the precipitating event may be administration of chemotherapeutic agents. In a further embodiment, the precipitating event may be the subject's admission to a hospital intensive care unit. In another embodiment, the precipitating event may be the subject developing infection-induced inflammation (sepsis).

In one embodiment, an amount effective of pyridoxamine, or a salt thereof, may be administered before (for example, 7 days, 6 days, 5 days, 4 days, 3 days, 2 days, and/or 1 day before) a precipitating event, or at the time of a precipitating event, or within 24 hours after a precipitating event (i.e.: within 24 hours, 23 hours, 22 hours, 21 hours, 20 hours, 19 hours, 18 hours, 17 hours, 16 hours, 15 hours, 14 hours, 13 hours, 12 hours, within 11 hours, within 10 hours, within 9 hours, within 8 hours within 7 hours, within 6 hours, within 5 hours, within 4 hours, within 3 hours, within 2 hours, or within 1 hour) and may continue to be administered following the precipitating event.

In another aspect, the invention provides methods of limiting development of acute kidney injury (AKI), comprising administering to a subject at risk of AKI an amount effective of pyridoxamine, or a pharmaceutically acceptable salt thereof, to limit development of the AKI.

In various embodiments, the risk factor for AKI includes, but is not limited to, low blood volume, infection-induced inflammation (sepsis), liver cirrhosis, renal artery stenosis, renal vein thrombosis, glomerulonephritis, acute tubular necrosis (ATN), acute interstitial nephritis (AIN), benign prostatic hyperplasia, exposure to an obstructed urinary catheter, bladder stone; and bladder, ureteral or renal malignancy. An amount effective of pyridoxamine, or a salt thereof, may be administered to a subject with a risk factor for AKI, and continue to be administered if the subject progresses to AKI.

In each of these aspects, embodiments, and combinations thereof, “limiting development of AKI” means any clinical benefit for the subject compared to a subject not treated with the methods of the invention (“control”). In various embodiments, limiting development of AKI may result in one or more of the following compared to control:
Limiting the increase in serum creatinine levels characteristic of AKI;
Limiting the decrease in glomerular filtration rate characteristic of AKI;
Reducing the decrease in urine volume characteristic of AKI;
Limiting the renal fibrosis characteristic of AKI;
Limiting development of one or more other symptoms of AKI, including but not limited to metabolic acidosis, high potassium levels (and potentially resulting irregular heartbeat), uremia, changes in body fluid balance, and effects to other organ systems;
Limiting progression to chronic renal disease;
Limiting need for renal dialysis; and
Limiting need for kidney transplant.

In each of these aspects, embodiments, and combinations thereof, pyridoxamine or a pharmaceutically acceptable salt thereof is administered to the subject prior to onset of AKI. As will be understood by those of skill in the art, the pyridoxamine or salt thereof may continue to be administered after onset of AKI, as deemed appropriate by an attending physician.

In another aspect, the invention provides method of treating development of acute kidney injury (AKI), comprising administering to a subject with AKI an amount effective of pyridoxamine, or a pharmaceutically acceptable salt thereof, to treat AKI.

In this aspect, “treating AKI” means any clinical benefit for the subject compared to a subject not treated with the methods of the invention (“control”). In various embodiments, treating AKI may result in one or more of the following compared to control:
Reducing or limiting the increase in serum creatinine levels characteristic of AKI;
Increasing or limiting the decrease in glomerular filtration rate characteristic of AKI;
Reducing the decrease in urine volume characteristic of AKI;
Limiting the renal fibrosis characteristic of AKI;
Limiting development of one or more other symptoms of AKI, including but not limited to metabolic acidosis, high potassium levels (and potentially resulting irregular heartbeat), uremia, changes in body fluid balance, and effects to other organ systems;
Limiting progression to chronic renal disease;
Limiting need for renal dialysis; and
Limiting need for kidney transplant.

In all aspects, embodiments and combinations of embodiments of the invention, the pyridoxamine, or salt thereof, may be administered at any frequency deemed appropriate by an attending physician (1× per day, 2× per day, every other day, etc.). Dosage unit forms for use in the present invention may comprise any suitable dosage of pyridoxamine or salt thereof as deemed appropriate by an attending physician. In non-limiting embodiments, the dosage units comprise between 1 mg and 1000 mg of pyridoxamine, or a pharmaceutically acceptable salt thereof. Such dosage unit forms can comprise, for example, between 1 mg-750 mg, 1 mg-500 mg, 1 mg-250 mg, 1 mg-100 mg, 50 mg-1000 mg, 50 mg-750 mg, 50 mg-500 mg, 50 mg-250 mg, 50 mg-100 mg, 100 mg-1000 mg, 100 mg-750 mg, 100 mg-500 mg, 100 mg-250 mg, 250 mg-1000 mg, 250 mg-750 mg, 250 mg-500 mg, 500 mg-1000 mg, 500 mg-750 mg, or 750 mg-1000 mg of pyridoxamine, or a pharmaceutically acceptable salt thereof. The dosage unit form can be selected to accommodate the desired frequency of administration used to achieve a specified daily dosage of pyridoxamine, or a pharmaceutically acceptable salt thereof to a subject in need thereof.

In all embodiments and combinations of embodiments of the invention, the subject may be any suitable subject including a mammalian subject, such as a human subject,

Pharmaceutically acceptable salts in accordance with the present invention are the salts with physiologically acceptable bases and/or acids well known to those skilled in the art of pharmaceutical technique. Suitable salts with physiologically acceptable bases include, for example, alkali metal and alkaline earth metal salts, such as sodium, potassium, calcium and magnesium salts, and ammonium salts and salts with suitable organic bases, such as methylamine, dimethylamine, trimethylamine, piperidine, morpholine and triethanolamine. Suitable salts with physiologically acceptable acids include, for example, salts with inorganic acids such as hydrohalides (especially hydrochlorides or hydrobromides), sulphates and phosphates, and salts with organic acids.

The pyridoxamine or salt thereof can be administered as a pharmaceutical formulation including those suitable for oral (including buccal and sub-lingual), rectal, nasal, topical, pulmonary, vaginal or parenteral (including intramuscular, intraarterial, intrathecal, subcutaneous and intravenous) administration or in a form suitable for administration by inhalation or insufflation. In various embodiments, the manner of administration is intravenous or oral (or alternative mucosal delivery, such as vaginal or nasal routes) using a convenient daily dosage regimen that can be adjusted according to the degree of affliction.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, and the like, an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known, or will be apparent, to those skilled in this art; for example, see Remington's Pharmaceutical Sciences, referenced above.

In yet another embodiment is the use of permeation enhancer excipients including polymers such as: polycations (chitosan and its quaternary ammonium derivatives, poly-L-arginine, aminated gelatin); polyanions (N-carboxymethyl chitosan, poly-acrylic acid); and, thiolated polymers (carboxymethyl cellulose-cysteine, polycarbophil-cysteine, chitosan-thiobutylamidine, chitosan-thioglycolic acid, chitosan-glutathione conjugates).

For oral administration, the composition will generally take the form of a tablet, capsule, a softgel capsule or can be an aqueous or nonaqueous solution, suspension or syrup. Tablets and capsules are preferred oral administration forms. Tablets and capsules for oral use can include one or more commonly used carriers such as lactose and corn starch. Lubricating agents, such as magnesium stearate, are also typically added. Typically, the compounds of the present disclosure can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

When liquid suspensions are used, the active agent can be combined with any oral, non-toxic, pharmaceutically acceptable inert carrier such as ethanol, glycerol, water, and the like and with emulsifying and suspending agents. If desired, flavoring, coloring and/or sweetening agents can be added as well. Other optional components for incorporation into an oral formulation herein include, but are not limited to, preservatives, suspending agents, thickening agents, and the like.

Parenteral formulations can be prepared in conventional forms, either as liquid solutions or suspensions, solid forms suitable for solubilization or suspension in liquid prior to injection, or as emulsions. Preferably, sterile injectable suspensions are formulated according to techniques known in the art using suitable carriers, dispersing or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Parenteral administration includes intraarticular, intravenous, intramuscular, intradermal, intraperitoneal, and subcutaneous routes, and include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Administration via certain parenteral routes can involve introducing the formulations of the present disclosure into the body of a patient through a needle or a catheter, propelled by a sterile syringe or some other mechanical device such as a continuous infusion system. A formulation provided by the present disclosure can be administered using a syringe, injector, pump, or any other device recognized in the art for parenteral administration.

Sterile injectable suspensions can be formulated according to techniques known in the art using suitable carriers, dispersing or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained. Preparations according to the present disclosure for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain adjuvants such as preserving, wetting, emulsifying, and dispersing agents. They can be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

The formulations can optionally contain an isotonicity agent. The formulations preferably contain an isotonicity agent, and glycerin is the most preferred isotonicity agent. The concentration of glycerin, when it is used, is in the range known in the art, such as, for example, about 1 mg/mL to about 20 mg/mL.

The pH of the parenteral formulations can be controlled by a buffering agent, such as phosphate, acetate, TRIS or L-arginine. The concentration of the buffering agent is preferably adequate to provide buffering of the pH during storage to maintain the pH at a target pH±0.2 pH unit. The preferred pH is between about 7 and about 8 when measured at room temperature.

Other additives, such as a pharmaceutically acceptable solubilizers like Tween 20® (polyoxyethylene (20) sorbitan monolaurate), Tween 40® (polyoxyethylene (20) sorbitan monopalmitate), Tween 80® (polyoxyethylene (20) sorbitan monooleate), Pluronic F68® (polyoxyethylene polyoxypropylene block copolymers), and PEG (polyethylene glycol) can optionally be added to the formulation, and can be useful if the formulations will contact plastic materials. In addition, the parenteral formulations can contain various antibacterial and antifungal agents, for example, parabens, chlorobutanol, phenol, sorbic acid, thimerosal, and the like.

Sterile injectable solutions can be prepared by incorporating pyridoxamine or salt thereof in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Thus, for example, a parenteral composition suitable for administration by injection is prepared by stirring 1.5% by weight of active ingredient in 10% by volume propylene glycol and water. The solution is made isotonic with sodium chloride and sterilized.

In another aspect, the invention provides methods for monitoring efficacy of pyridoxamine therapy, comprising

(a) determining one or more of the following in a biological sample obtained from a subject receiving pyridoxamine therapy (a) expression level of Col3α1, (b) expression level of αSMA, (c) expression level of Kim1, (d) expression level of NGAL, and/or (e) isofuran-to-isoprostane ratio (IsoF/IsoP); and

(b) comparing the levels of markers determined in step (a) to a control;

wherein those subjects with a decreased level of one or more of the markers compared to control are responding to pyridoxamine therapy.

As shown in the examples herein, successful pyridoxamine therapy results in a decreased expression (mRNA and/or protein) of Col3α1, Col1α1, αSMA, Kim1, and NGAL, and in a decreased isofuran-to-isoprostane ratio compared to control (ex: similar subjects not treated to pyridoxamine; pre-existing standards for expression levels or IsoF/IsoP ratios; etc.) Thus, the methods can be used to monitor efficacy in subjects receiving pyridoxamine therapy, such as pyridoxamine therapy for AKI, diabetic nephropathy, or other indications. Any suitable biological sample can be used, including but not limited to a kidney biopsy, a blood sample, etc.

In one embodiment, the steps can be carried out more than once (2, 3, 4, 5, 6, or more times) to monitor treatment progression over time. In a further embodiment, a subsequent pyridoxamine dosage may be increased if the subject is determined as not having decreased level of one or more of the markers compared to control.

In one embodiment, the markers determined include at least the isofuran-to-isoprostane ratio.

### EXAMPLES

Two doses of pyridoxamine were administered orally, 500 mg/kg/day and 1000 mg/kg/day to an experimental model of AKI, the surgical, ischemia-reperfusion model of AKI in mice (IR-AKI) [Cianciolo Cosentino et al, 2013; Skrypnyk et al, 2013], a model of renal ischemia that has been used extensively to model ischemic injury associated with cardiac surgery acquired (CSA-AKI) [Thiele et al, 2015]. For the bulk of the studies pyridoxamine was administered for 3 days prior to the induction of AKI and was continued until completion of the studies. In some experiments, pyridoxamine was administered 24 hours after the induction of AKI.

### Example 1

### Dose Dependent Effects of Pre-Treatment with Pyridoxamine at 500 and 1000 mg/kg/Day on Renal Fibrosis 28 Days after I/R-AKI

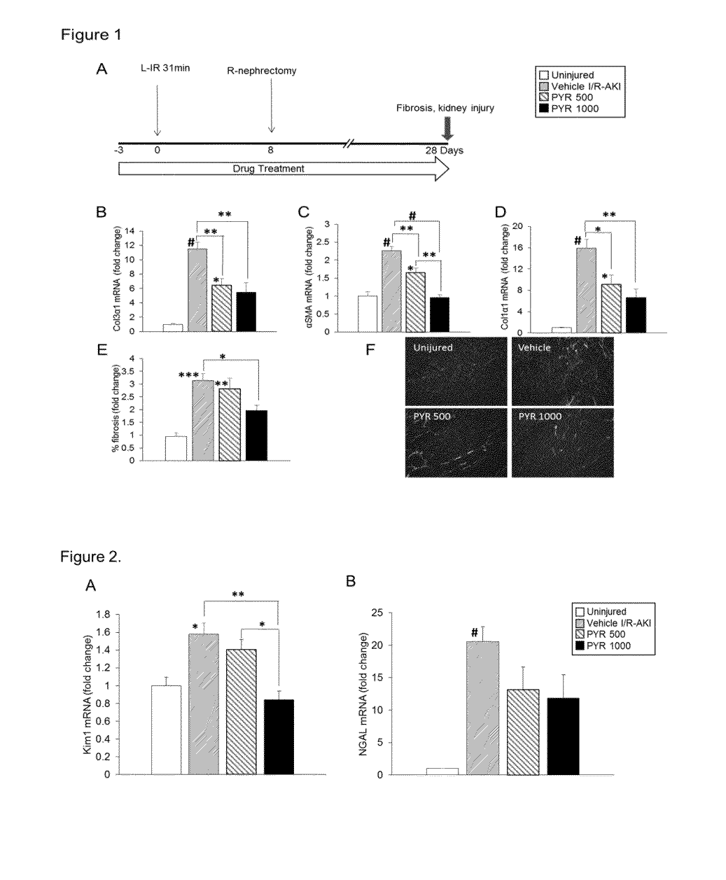

The injury models and treatments were administered as illustrated in FIG. 1A. Mice underwent unilateral renal pedicle clamping (U-IR) followed by contralateral nephrectomy 8 days after the initial surgery. All mice were pre-treated for 3 days with either vehicle control, or PYR 500 and 1000 mg/kg/day in drinking water supplemented with 200 mg PYR twice a day (or vehicle) by oral gavage for 3 days after each surgical procedure. Treatment was continued for 28 days at which point mice were sacrificed and kidneys harvested for analysis. Pre-treatment with pyridoxamine at 500 and 1000 mg/kg lowered expression of pro-fibrotic genes Col3α1, αSMA and Col1α1 mRNAs (FIG. 1, B-D) and decreased level of fibrosis (FIGS. 1, E and F) in a dose dependent manner.

### Example 2

### Dose Dependent Effects of Pre-Treatment with Pyridoxamine at 500 and 1000 mg/kg/Day on Markers of Renal Injury 28 Days after I/R-AKI

Markers of renal injury were evaluated 28 days after the initiation of injury following the dosing regimen shown in FIG. 1A. Pre-treatment with pyridoxamine at 500 and 1000 mg/kg/day lowered expression of renal injury marker Kim1 (FIG. 2A) but not NGAL (FIG. 2B) on day 28 after injury.

### Example 3

### Beneficial Effects of Treatment with Pyridoxamine at 1000 mg/kg/Day Started 24 Hours after Injury on Renal Fibrosis 28 Days after I/R-AKI

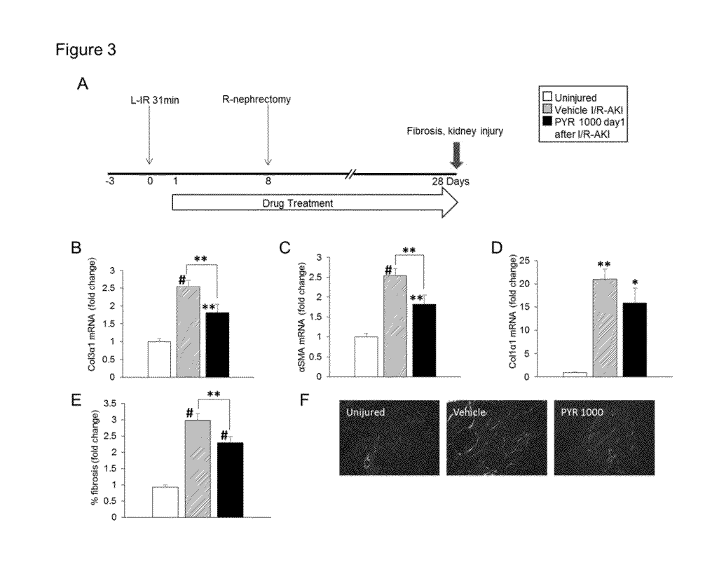

To determine whether delayed treatment with the high dose of pyridoxamine was effective in reducing post-injury fibrosis after IR-AKI, mice were treated with PYR 1000 mg/kg/day starting 24 hours after the initial injury supplemented with 200 mg PYR twice a day (or vehicle) by oral gavage for 3 days after each surgical procedure. Treatment was continued for 28 days at which point mice were sacrificed and kidney harvested for analysis (FIG. 3A). Delayed pyridoxamine treatment started 24 hours after injury lowered expression of pro-fibrotic genes Col3α1 and αSMA (FIGS. 3B and C) but not Col1α1 (FIG. 3D) mRNAs and decreased post-injury fibrosis (FIGS. 3E and F).

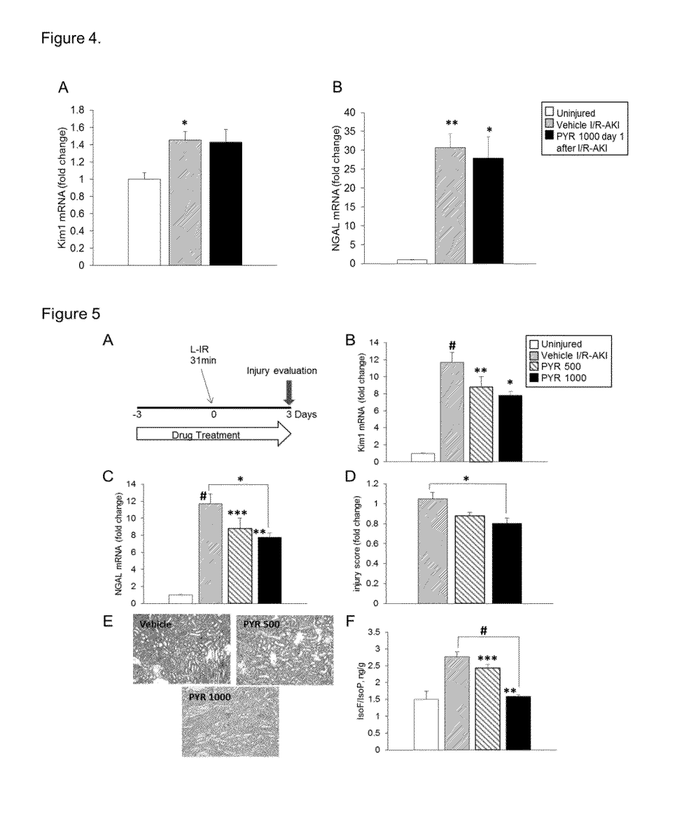

Delayed pyridoxamine treatment started 24 hours after injury did not lower expression of injury markers Kim1 and NGAL on day 28 after injury (FIGS. 4A and B).

These data indicate that: a) delayed treatment with pyridoxamine at 1000 mg/kg/day had beneficial effect on renal fibrosis 28 days after the initiating AKI injury; and b) pre-treatment with pyridoxamine is more effective in reducing chronic renal injury after I/R-AKI compared to delayed treatment.

### Example 4

### Dose Dependent Effect of Pretreatment with Pyridoxamine at 500 and 1000 Mg/Kg/Day) on Markers of Renal Injury and Oxidative Stress 3 Days after I/R-AKI

To determine whether there was also a dose-dependent effect of pyridoxamine at 500 and 1000 mg/kg/day on early renal injury after IR-AKI, mice were sacrificed on day 3 after injury (FIG. 5A) to evaluate renal injury and renal oxidative stress levels. There was a significant, dose-dependent decrease in renal NGAL but not Kim 1 mRNA expression (FIGS. 5B and C), reduction in histological tubular injury scores (FIGS. 5D and E) and reduction in renal levels of oxidative stress marker isofuran-to-isoprostane ratio (FIG. 5F) in mice treated with 1000 mg/kg/day but not 500 mg/kg/day pyridoxamine compared with mice treated with the vehicle.

These data indicate that there is a reduction in early renal injury after IR-AKI in mice treated with pyridoxamine. The data also indicate that pyridoxamine at 1000 mg/kg/day is more effective than 500 mg/kg/day at reducing early renal injury after IR-AKI.

### Example 5

### Plasma Pyridoxamine Levels after I/R-AKI

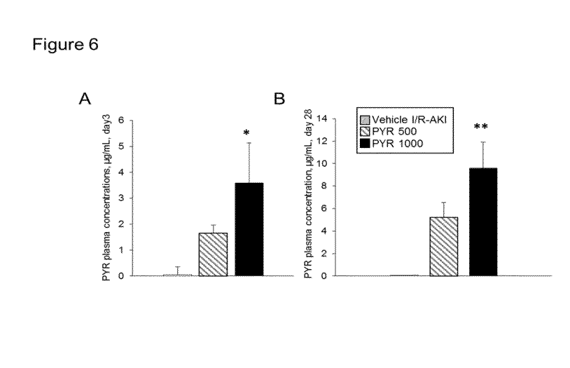

Having established that 1000 mg/kg/day Pyridorin is more effective in preventing early and long term kidney injury after IR-AKI in mice, plasma levels of pyridoxamine were determined in mice with IR-AKI treated with 500 mg/kg/day and 1000 mg/kg/day pyridoxamine for 3 and 28 days (FIG. 6). At each time point, there was a dose-dependent increase in plasma pyridoxamine levels (FIG. 6). In mice treated with pyridoxamine at 1000 mg/kg/day, the average pyridoxamine plasma levels on days 3 and 28 were ˜6 μg/mL (FIGS. 6A and B), thus suggesting that at these plasma levels pyridoxamine is therapeutically effective in mouse IR-AKI.

We claim:

1. A method of limiting development of acute kidney injury (AKI), comprising administering to a subject to be subjected to a precipitating event an amount effective of pyridoxamine, or a pharmaceutically acceptable salt thereof, to limit development of the AKI, wherein the administering comprises administering pyridoxamine, or a pharmaceutically acceptable salt thereof, to the subject prior to, at the time of, or within 24 hours of the precipitating event.

2. The method of claim 1 wherein the precipitating event is selected from the group consisting of cardiovascular surgery, injection of a contrast dye, administration of chemotherapeutic agents, development of an infection-induced inflammation (sepsis), and admission to a hospital intensive care unit.

3. A method of limiting development of acute kidney injury (AKI), comprising administering to a subject at risk of AKI an amount effective of pyridoxamine, or a pharmaceutically acceptable salt thereof, to limit development of the AKI.

4. The method of claim 3, wherein the subject has a risk factor for AKI selected from the group consisting of low blood volume, liver cirrhosis, infection-induced inflammation (sepsis), renal artery stenosis, renal vein thrombosis, glomerulonephritis, acute tubular necrosis (ATN), acute interstitial nephritis (AIN), benign prostatic hyperplasia, exposure to an obstructed urinary catheter, bladder stone; and bladder, ureteral or renal malignancy.

5. The method of claim 1, wherein limiting the development of AKI comprises one or more of the following:
Limiting the increase in serum creatinine levels characteristic of AKI;
Limiting the decrease in glomerular filtration rate characteristic of AKI;
Reducing the decrease in urine volume characteristic of AKI;
Limiting the renal fibrosis characteristic of AKI;
Limiting development of one or more other symptoms of AKI, including but not limited to metabolic acidosis, high potassium levels (and potentially resulting irregular heartbeat), uremia, changes in body fluid balance, and effects to other organ systems;
Limiting progression to chronic renal disease;
Limiting need for renal dialysis; and
Limiting need for kidney transplant.

6. The method of claim 3, wherein limiting the development of AKI comprises one or more of the following:
Limiting the increase in serum creatinine levels characteristic of AKI;
Limiting the decrease in glomerular filtration rate characteristic of AKI;
Reducing the decrease in urine volume characteristic of AKI;
Limiting the renal fibrosis characteristic of AKI;
Limiting development of one or more other symptoms of AKI, including but not limited to metabolic acidosis, high potassium levels (and potentially resulting irregular heartbeat), uremia, changes in body fluid balance, and effects to other organ systems;
Limiting progression to chronic renal disease;
Limiting need for renal dialysis; and
Limiting need for kidney transplant.

7. A method of treating development of acute kidney injury (AKI), comprising administering to a subject with AKI an amount effective of pyridoxamine, or a pharmaceutically acceptable salt thereof, to treat AKI.

8. The method of claim 7, wherein treating AKI comprises one or more of:
Reducing or limiting the increase in serum creatinine levels characteristic of AKI;
Increasing or limiting the decrease in glomerular filtration rate characteristic of AKI;
Reducing the decrease in urine volume characteristic of AKI;
Limiting the renal fibrosis characteristic of AKI;
Limiting development of one or more other symptoms of AKI, including but not limited to metabolic acidosis, high potassium levels (and potentially resulting irregular heartbeat), uremia, changes in body fluid balance, and effects to other organ systems;
Limiting progression to chronic renal disease;
Limiting need for renal dialysis; and
Limiting need for kidney transplant.

9. The method of claim 1, wherein the pyridoxamine or pharmaceutically acceptable salt thereof is administered to the subject at least once per day at a dosage unit of between 1 mg/kg and 1000 mg/kg.

10. A method for monitoring efficacy of pyridoxamine therapy, comprising
(a) determining one or more of the following in a biological sample obtained from a subject receiving pyridoxamine therapy (a) expression level of Col3α1, (b) expression level of αSMA, (c) expression level of Kim1, (d) expression level of NGAL, (e) expression level of Col1α1, and/or (e) isofuran-to-isoprostane ratio (IsoF/IsoP); and
(b) comparing the levels of markers determined in step (a) to a control;
wherein those subjects with a decreased level of one or more of the markers compared to control are responding to pyridoxamine therapy.

11. The method of claim 10, wherein the subject has AKI.

12. The method of claim 10, wherein a subsequent dosage of pyridoxamine, or a pharmaceutical salt thereof, to the subject is increased if the level of the one or more markers is not increased in the biological sample.

13. The method of claim 9, wherein the biological sample comprises a kidney biopsy.

14. The method of claim 1, wherein the subject is a mammal.

15. The method of claim 1, wherein the subject is a human.
